(19) United States
(12) Patent Application Publication
(10) Pub. No.: US 2021/0140981 A1
(43) Pub. Date: May 13, 2021

(54) BLOOD READER SYSTEMS AND THERONOSTICS FOR BRAIN DAMAGE AND INJURY

(71) Applicant: Barry W. FESTOFF, Kansas City, MO (US)

(72) Inventor: Barry W. FESTOFF, Kansas City, MO (US)

(21) Appl. No.: 17/046,606

(22) PCT Filed: Apr. 9, 2019
(86) PCT No.: PCT/US2019/026603

§ 371 Date: Oct. 9, 2020

Related U.S. Application Data
(60) Provisional application No. 62/654,779, filed on Apr. 9, 2018.
(60) Provisional application No. 62/660,701, filed on Apr. 20, 2018.

(51) Int. Cl. G01N 33/68; C12Q 1/6883; C12N 15/85; C07K 14/745; C07K 14/47; A61K 38/00; A61B 5/157; A61B 5/151; G16H 10/40

(52) U.S. Cl. CPC G01N 33/6896; A61B 5/150022; C12N 15/85; C07K 14/7455; C07K 14/4702; A61K 38/00; A61B 5/157; A61B 5/151; G16H 10/40; G01N 2333/4703; G01N 2800/28; G01N 2800/52; C12Q 2600/118; C12N 2800/22; C07K 2319/055; C12Q 1/6883

(57) ABSTRACT

Blood and bodily fluid reader systems, including circulating biomarkers involving multiple mitochondrial releasates for providing real-time, at-the-scene objective indicia of individuals sustaining mild TBI. As a therapeutic component for related neuroin-flammation of mild TBI and related injuries, medicaments, compounds and methods include chimeric proteins which combine: (i) a first polypeptide sequence derived from the “Box A” domain of the “High-Mobility Group Box 1” Protein ( HMGB1) protein; and, (ii) a second polypeptide sequence derived from the D1 lectin-like domain of thrombomodulin (TM). These chimeric proteins both activate and promote certain repair-type functions within the brain, up to and in some cases including the growth of new neuronal fibers and/or the creation of new synaptic junctions; and, keep those types of inflammation-triggered repair processes within healthy and desirable limits.

### CROSS-REFERENCE TO RELATED APPLICATIONS

This application claims priority to provisional application, Ser. No. 62/654779 filed Apr. 9, 2018 and provisional application Ser. No. 62/660701 filed Apr. 20, 2018, which are incorporated herein by reference.

### STATEMENT REGARDING FEDERALLY SPONSORED RESEARCH

Not Applicable.

### SEQUENCE LISTING STATEMENT

The official copy of the sequence listing is submitted electronically via EFS-Web as an ASCII formatted sequence listing with a file named “______”, created on ______, and having a size of “______”. The sequence listing contained in this ASCII formatted document is part of the specification and is herein incorporated by reference in its entirety.

### FIELD OF THE INVENTION

The subject matter herein relates generally to the field of neurology and medical technology, and more specifically to blood reader systems, including circulating biomarkers and therapeutics based on such a system for concussions or comparable types of head trauma or brain injuries.

### BACKGROUND OF THE INVENTION

Within the medical profession, injuries to the head and/or brain, which are caused by forceful impacts are classified as “mild” (even though some such injuries may lead to serious and even severe long-term damage), if certain types of indicators are not present which would otherwise indicate a need for immediate hospitalization and critical-care intervention. If present they are designated moderate or severe.

According to the U.S. Centers for Disease Control and Prevention (CDC), mild traumatic brain injury (mTBI), which is often used interchangeably with concussion, likely affects between 1.5 and 2 million people annually, in the U.S. The CDC figures are based on individuals that actually seek medical attention for their TBI. Since most mTBI/concussed persons do not seek medical attention the number may be 4-5 times higher. The CDC also estimates that between 3.5 and 4 million people annually suffer from the types of moderate and severe TBI. The CDC goes on to state that TBI is caused by a bump, blow, or jolt to the head that disrupts the normal function of the brain, but not all blows or jolts to the head result in a TBI, these are usually termed sub-concussive hits. The objective diagnosis and related treatment of mTBI and/or concussion is the basis for this invention. The CDC further estimate that 15 to 20% of the annual TBI cases will go on to post-concussion syndrome (PCS), usually within 7 to 10 days after the concussion; about 50% of those individuals will continue to suffer from symptoms of PCS three months after the head trauma, and 20% will still have symptoms a full year after the injury.

Trained specialists who work in hospital admitting rooms use an evaluation system that assigns a “Glasgow Coma Score” (GCS) to arriving patients. However, those types of evaluations, by trained experts, are reached only after a patient has been taken to a hospital. Accordingly, a major need has arisen for better methods that will allow police, firemen, athletic coaches and trainers, ambulance attendants, and other semi-trained laypersons to accurately and reliably evaluate what appears to be a “mild” head injury, at the site, or venue, where an accident or other similar event occurred.

Traditional diagnosis of a concussion, or a mild TBI (mTBI), is poor and inaccurate, and even at best, diagnosis of borderline cases (which are very frequent) is heavily subjective as described below. Over the last decade and a half, intensive efforts towards defining objective and clinically-useful biomarkers have not been successful, in large part because the blood-brain barrier (BBB) is very efficient at keeping most types of extracellular molecules either inside the brain, or outside the brain.

Accordingly, it would be highly useful if coaches and trainers, on collegiate and professional sports teams, and at any other level, equally or even more critically, such as high school, junior high school, youth sports organizations (and in any sport which poses a risk of serious collisions or violent impacts), could have and use a simple and convenient diagnostic tool and method, which could provide a valid and reliable indicator of m TBI.

Beyond those types of sports-related injuries, numerous other types of trained personnel (including police, firemen, ambulance attendants, and even teachers and various types of caregivers) encounter situations where they witness (or are called upon as a “first-responder” to) an accident, which may involve a concussion or similar mTBI,

Therefore, if diagnostic tools and methods were available which could provide coaches, trainers, police, firemen, and the owners of places (such as ski resorts) where head traumas are likely to occur, with an objective capability of evaluating the true severity of a head trauma, it could save lives, reduce costs, and prevent terrible suffering, including the suffering of family members and loved ones who lost (or who must spend the rest of their lives caring for) someone who was young, healthy, athletic, and promising, up until the day of an accident.

Moreover, it has been estimated that, in the U.S., from one- to two-thirds of TBI patients whose head injuries were sufficiently severe or worrisome to cause them to go to an hospital, are subsequently assessed as “mild” TBI or concussion patients, and are sent home within a few hours after arriving at the hospital (or after an overnight stay, if they arrive in the evening or nighttime), with no special medical treatments that required a prescription or active involvement from a doctor. They are advised to contact their primary clinical provider (PCP) after several weeks or sooner if their symptoms get worse.

Most of those patients recover, without significant short or mid-term consequences. However, substantial numbers of such patients develop persistent neurological, behavioral, and cognitive symptoms, which often include, for example, recurring headaches, memory disturbances, difficulties in concentrating, and lingering anxiety and/or depression). These types of symptoms, if they arise following a concussion or mild TBI, are often generally lumped together, and are referred to as “post-concussion syndrome” (PCS). It has been estimated that: (i) than three-quarters of all concussion and mild TBI patients will have one or more symptoms that indicate PCS up to 3 months after the concussion or; mTBI and, (ii) 15% of such patients will continue to have one or more of such symptoms, a full year or more after the accident, considered “persistent” (pPCS). Furthermore, the medical disability and insurance claims, and occupational problems (including frequent requests for time off from work) that are associated with pPCS, are quite sizeable, and impose large economic burdens on society, employers, and workers.

For the foregoing reasons, it is not sufficient for people such as football coaches, policemen, or ambulance attendants to create and then rely upon a “single-moment-in-time” analysis of a head trauma, since that “snapshot” type of single-instant evaluation cannot adequately determine or foretell which players or patients are at substantially elevated risk of a serious neurologic problem that may not begin to seriously manifest and become apparent until more time has passed after a “blow to the head”.

Accordingly, one object of this invention is to establish a blood reader system, device and method including a set, or panel, of biochemical markers in the blood, which will reliably act as a predictor of the type and severity of the type of neuronal injury that is most likely to occur, during the hours following a blow to the head.

Another object of this invention is to establish such a testing device and system which will use specialized and disposable diagnostic components that have been prepared in advance and that are designed to interact with the testing device, and which can receive a small sample of blood or saliva, taken from the player, patient, or victim several minutes after the head trauma, and which analyze in real-time, or in short order, a set of biochemical markers that provide a useful predictive indicator of how severe the cellular and neuronal damage actually was, inside the brain, and which indicates whether a player, patient, or victim needs immediate medical attention, to prevent additional and possibly permanent brain damage.

Related to the need for more reliable determination of mTBI, is that for injectable drugs that can reduce brain damage after a head injury or similar crisis, and which also can be used to treat various types of ongoing or chronic neuro-inflammatory problems. These drugs may involve “chimeric” proteins, which contain active fragments derived from completely different proteins. By coupling together different fragments obtained from different proteins which have different activities, a “chimeric” protein may be created which can offer new and useful treatments for injured, infected, or otherwise damaged brain or spinal tissue.

### The HMGB1 Protein

HMGB1 is an acronym that refers to “high mobility group box 1”, a protein belonging to a family of high mobility group ( HMG) proteins, so-called because of high electrophoretic mobility in polyacrylamide gels. It has two completely different sets of functions, depending on whether it is inside a cell, or outside a cell. When kept inside a cell, HMGB1 remains in the nucleus, where it becomes one of the most important proteins that interact with chromosomal DNA. Accordingly, intra-cellular HMGB1 is a “chromatin” protein, where “chromatin” refers to the entire mass of DNA and associated proteins (including histone proteins, transcription proteins, gene-regulating proteins, etc.) that are contained in the nucleus of a cell. HMG proteins are the most ubiquitous non-histone proteins associated with chromatin, in mammalian cells, and they play crucially important roles in bending, looping, folding and other actions that handle and manipulate the genes of a cell.

By contrast, when HMGB1 is released by dying cells or secreted by living cells, it can become a serious and even severe trouble-maker, which can trigger and aggravate inflammation in ways that are not wanted, helpful, or useful. In general, inflammation (including neuro-inflammation) is a protective response to various types of cell and tissue injuries and infections, and HMGB1 participates in those. That type of release of HMGB1, by cells, occurs when certain types of immune cells (including macrophages and monocytes) are contacted by certain types of triggering agents; it will also occur when cells are killed and ruptured, due to injury, infection, or disease (“necrosis”, as distinct from the controlled recycling of old and senescent cells by a process called apoptosis).

### Neuro-Inflammation

As used herein, the term “neuro-inflammation” is limited to “inflammation” that occurs within the central nervous system (CNS; i.e., within the brain and/or spinal cord). In other words, “neuro-inflammation” (as used herein) must directly affect neurons and/or glial cells in CNS tissue, in order to be covered by the discussion or claims herein. Neurons outside the CNS (which includes neurons of the “peripheral” and/or “sympathetic” nervous systems) may also be affected, in some cases; however, the test for determining whether “neuro-inflammation” (as addressed and covered by this invention) is occurring depends on whether neurons and/or glial cells inside the brain or spinal cord are being directly affected.

As a point of clarification, some CNS neurons have their main cell bodies inside the CNS, but also have specialized fibers (often called dendrites or similar terms) that extend outside the brain and spinal cord. For all purposes herein, the test of whether a neuron is located inside or outside the CNS depends on the location of the main neuronal cell body (which will necessarily include the cell nucleus), regardless of whether one or more neuronal fibers extend outside the CNS tissue.

The reference above to “glial cells” also requires comment. That term refers to and includes any cells in brain or spinal cord tissue (excluding cells in blood vessels) that do not send or receive nerve signals. The term “glial” comes from the same root word as “glue”; glial cells were given that name before their functions were understood, when it was assumed that they merely supplied a supporting matrix which helped neurons create and position the long fibers they use to communicate with each other. It is now known that there are several major classes of glial cells, and they provide a number of crucially important “housekeeping and support” functions for neurons. As one example, glial cells called “oligodendrocytes” create and maintain the “myelin sheaths” that surround neuronal fibers. Those “myelin sheaths” enable the electrochemical surges which create nerve signals to travel through the myelin-coated neuronal fibers, in a manner which is directly comparable to the way that a layer of non-conductive plastic, around a metal wire, enables electrical currents to pass through the coated and insulated wire without being lost to surrounding points of contact.

A second group of glial cells are astrocytes, so-called because of their star-shaped appearance in microscopic sections of CNS tissue. They are a critically important class of glial cells, which play fundamental roles as support cells for neurons, and in helping regulate energy supplies and transfers inside the brain. “Astrogliosis” often becomes an important factor during or after an injury to the brain or spinal cord, and it often becomes part of a neuro-inflammatory process that, in many cases, makes the pathology worse, and leads to progressive neurodegeneration. Astrocytes also interact closely with endothelial (i.e., blood vessel wall) cells inside the CNS, to form the blood-brain barrier (BBB), a cellular barrier that controls the vertebrate brain's internal environment. This close relationship between astrocytes and endothelial cells becomes even closer, during situations of oxidative stress and neuro-inflammation. In these situations, and especially after a traumatic brain injury (TBI), some of the cellular factors that are released by astrocyte cells can cause increased permeability of the BBB, in ways that can allow unwanted molecules to pass through the BBB, to make the crisis and the damage even worse.

As a third example, another class of glial cells which are directly relevant to this invention, as described below, are called “microglial” cells, or microglia, since they are relatively small (in comparison to neurons), and do not have any fibrous projections. Their small size, combined with the absence of fibers, makes it much easier for microglia to travel and migrate through brain tissue. That is an essential trait, since microglia create what is, in effect, as the CNS version of an immune system. Rather than generating or using antibodies, which will effectively label an invading pathogen in a way that causes it to be engulfed and destroyed by a macrophage (which is a complex type of immune cell), the brain and spinal cord use microglia to attack and in many cases surround anything which is interpreted to be “foreign” by the brain or spinal cord. In most cases, numerous microglia, acting in concert with each other, will attack, and will gradually dissolve and digest, whatever they have surrounded; however, it takes a relatively long time for them to do so, in comparison to how a single macrophage cell outside the brain or spinal cord can rapidly engulf, surround, devour, and digest large numbers of bacterial invaders or virus particles. In other cases, if a cluster or pocket of microbes forms, inside the brain or spinal cord, or if a foreign particle becomes lodged inside brain or spinal tissue after an injury or trauma, a layer of microglia can surround that pocket or particle, and then effectively die or become dormant, in a way which effectively isolates and smothers any pathogens inside a coating or shell of dead, dormant, or inert glial cells; this process is called “reactive gliosis”.

Glial cells can aggravate neuro-inflammation, in several ways. For example, in response to certain types of stress or infection, glial cells (especially astrocytes) can swell up to abnormally large sizes, due to excessive uptake of the clear liquid called cerebrospinal fluid (CSF). The medical term for excessive fluid accumulation, which leads to cell or tissue swelling is “edema”, regardless of where it occurs in a mammalian body. If edema begins to occur inside the brain, it can become extremely dangerous and even lethal, since swelling of cells or tissue inside the skull casing will lead to increased fluid pressures, which will begin pressing against the outer walls of blood vessels inside the brain. The walls of capillaries in particular are very thin, in order to enable high levels of nutrient and metabolite transfers into and out of the brain or spinal cord tissue. Therefore, those capillary walls cannot resist and push back, if elevated fluid pressures begin to press against their outer surfaces. If circulating blood encounters greater resistance (i.e., abnormally high fluid pressure) inside the brain, the blood will follow the basic laws of fluid mechanics, and will be diverted toward any available paths of lower resistance, thereby depriving the brain of the blood it needs to function properly. Inadequate blood supply to the brain can trigger excitotoxic, neurotoxic, and other problems which can begin killing neurons and glial cells in large numbers, leading to permanent brain damage or even death. Accordingly, edema inside the brain and/or spinal cord (which involves any swelling of neurons and/or glial cells) is regarded herein as a type of neuro-inflammation.

eral other medical terms also deserve mention. For purposes herein, the terms “swelling” and “inflammation” are used interchangeably. The standard medical definition of “inflammation” in peripheral (outside the CNS) tissues, lists four “cardinal signs”, which are redness, heat/warmth, swelling, and pain (impaired function is also listed as a frequent fifth sign). However, not all of those signs must be present, in any particular case of inflammation, and this is especially true for neuroinflammation, a much more complex form. Accordingly, to simplify and clarify this discussion and analysis, any swelling of cells or tissue, if and when it reaches a point or a level of severity that would be interpreted by a skilled physician or neurologist as indicating a pathological condition (i.e., a level which is either causing or displaying a medical problem which should be treated by some sort of medical intervention) is regarded and referred to as “inflammation”, and the terms “swelling” and “inflammation” are used interchangeably, herein.

The suffix “-itis” indicates swelling and/or inflammation, as occurs in words such as appendicitis, hepatitis, pancreatitis, etc. Accordingly, since “cephalon” and “encephalon” refer to the brain, the term “encephalitis” refers to swelling/inflammation of brain tissue.

The membranes that surround the brain and/or spinal cord also are regarded as components of those organs, and if they become inflamed, they will directly affect brain and/or spinal cord tissue and neurons in ways that can be regarded and classified as neuro-inflammation. Accordingly, “meningitis” and “arachnoiditis” (i.e. swelling of the meningeal or arachnoidal membranes, which surround the brain and spinal cord) are regarded and treated herein as forms or types of neuro-inflammation.

n understood and approached in this manner, “neuroinflammation” encompasses a class of problems which can be caused or aggravated by a range and variety of different types of events, diseases, infections, or other conditions. Furthermore, it should be understood that “neuroinflammation” almost always arises as a result or effect of some other “primary” or “causative” event, problem, or factor, such as: (i) a head or spinal injury, near-suffocation, or other trauma; (ii) an infection, which may occur after an injury or other problem has given bacteria or viruses an opportunity to reach brain or spinal cord tissue; or, (iii) a progressive neurodegenerative disease, such as Alzheimer's, Parkinson's, etc., which can render the brain unable to maintain its normal homeostatic balances, set points, and/or equilibrium-seeking processes.

A major unmet medical need exists for a widely applicable but carefully targeted pharmaceutical compound which can specifically target the effects, symptoms, and manifestations of neuroinflammation, without causing unwanted disruption of other metabolic or bodily processes.

Accordingly, another object of this invention is to provide additional treatment options which can be used, either on an acute basis for a short period of time (at higher dosage levels), or over a longer period of time (at lower dosage levels), in ways that will specifically target and improve a neuro-inflammatory problem, in ways that can help a patient's normal and natural repair mechanisms deal with the problem, and either fix the problem, or at least make it less severe.

Accordingly, another object of this invention is providing medicaments and methods for treating neuroinflammation in human patients in need of such treatment, by using a chimeric protein which combines specific selected domains from different proteins.

Another object of this invention is provision of medicaments and methods for such treatments, by using genetic vectors, which carry genes that encode the chimeric proteins described herein, and which are designed to introduce those genes into CNS neurons that are inside brain or spinal tissue that is protected by the BBB.

These and other objects of the invention will become more apparent from the following summary, drawings, detailed description, and examples.

### SUMMARY OF THE INVENTION

Systems, methods, devices and materials are disclosed for athletic field, military, emergency-responder, or other on-site, in transit, emergency room, or similar evaluation of concussions and similar traumatic head and/or brain injuries. These blood reader systems, methods, devices and materials involve diagnostic bioreagents (such as monoclonal antibodies, single-stranded DNA or RNA, etc.) which are affixed to surfaces of computer-readable devices (such as compact discs, readable cards or strips, etc.) that are designed to be handled by electronic sensor devices that can interact with portable computers (such as laptop computers, computer pads or tablets, smart phones, portable digital assistants, etc.). The bioreagents are selected to detect the presence and concentration of metabolites released by mitochondria, preferably at least two selected metabolites released by mitochondria in response to severe cellular damage, preferably mitochondrial DAMPs (mtDAMPs) as releasates. In further embodiments, additional bioreagents may also be included, for detecting and quantifying one or more non-mitochondrial “damage-associated molecular patterns” (abbreviated as DAMPs). When used in conjunction with other available tests, which can include simple cognitive, reasoning, and/or response tests (as well as more complex analyses, if available), which the Applicant terms “clinimetrics,” this type of analysis, focusing upon metabolites released by mitochondria, can be used to assist coaches, trainers, military personnel, physicians, and others, to determine the nature and the severity of and devise proper responses and take steps to address head traumas and other conditions that otherwise can be very difficult or impossible to evaluate and act upon.

Blood and bodily fluid reader systems, including circulating biomarkers involving multiple mitochondrial releasates for providing real-time, at-the-scene objective indicia of individuals sustaining mild TBI are provided as an aspect of this invention. In particular, as further detailed below, a diagnostic reader system is provided including a portable reader unit designed to be carried by a user, and hand powered by a microprocessor and coupled to a portable computer, smart phone, etc. and configured to allow data transfer between the diagnostic reader and the portable computer, etc. In this diagnostic reader, a stylet or other unit is contained to obtain a blood sample by finger stick or other bodily fluid sample, which is operably connected to a disposable diagnostic device which has been contacted by the blood or other fluid sample taken from a person who may have suffered a blow or trauma involving the person's head. In this aspect, the disposable diagnostic device has: e.g., at least one first reactive surface which has a first type of biomolecular reagent bonded to it by which to measure a blood-borne concentration of at least one first mitochondrial releasate; at least one second reactive surface which has a second type of biomolecular reagent bonded to it by which to measure a blood-borne concentration of at least one second mitochondrial releasate; and, at least one third reactive surface which has a third type of biomolecular reagent bonded to it by which to measure a blood-borne concentration of at least one third mitochondrial releasate. In this embodiment, the diagnostic reader system has a data handler selected from the group consisting of: displaying, in a manner visible to a user, both of the blood-borne concentrations of the first, second and third mitochondrial releasates; and/or transferring, to a portable computer, such as a smartphone or tablet, which has a display monitor, the blood-borne concentrations of the first, second and third mitochondrial releasates. In a further embodiment, the first, second and third mitochondrial releasates are selected from the group consisting of: DNA segments (“native”) which are unaltered and specific to mitochondrial genes (mitochondrial DNA) and which do not normally occur in human nuclear DNA; fragments of mitochondrial DNA that have been degraded by oxidative radicals as a result of the brain injury in ways that normally are found, in humans, only in DNA fragments that have been released by mitochondria; proteins that are encoded by mitochondrial or nuclear DNA and concentrated in mitochondria prior to their rupture by the brain injury, including but not limited to,“high mobility group” ( HMG) proteins, High mobility group box 1 protein ( HMGB1), Transcription Factor for Mitochondria A (TFAM); Cytochrome C oxidase, Cyclophilin D, Subunit 6 of ATP synthase, N-formyl peptides (N-FPs) and formyl peptide receptors (FPRs).

In another aspect, the diagnostic reader system as described above may further involve position of the first and second reactive surfaces in different locations on the single disposable diagnostic device, separated from the third reactive surface. The diagnostic reader system as described herein, is also capable of reading data from different disposable diagnostic devices which have distinct areas that have been coated with biomolecular reagents that will indicate concentrations of one or more blood-borne human proteins selected from the group consisting of: apolipoprotein E, apolipoprotein A-1, one or more selected TAR DNA binding proteins, one or more cellular damage-associated molecular patterns (DAMPs) and damage-related proteins selected from the group consisting of: HMGB1 and TFAM (above as mitochondrial DAMPs) and, angiotensin-converting enzyme serpin proteins, and plasminogen activator inhibitors, cytokines and/or other proinflammatory mediators, mRNA from genes which encode subunits of receptors that interact with cytokines or proinflammatory mediators and that include thrombomodulin (THBD) endothelial cell protein C receptor (ECPCR), 5-hydroxytryptamine receptor 2A, the serotonin transporter (SERT) or solute carrier family 6 (neurotransmitter transporter, 5-HTT), the human protein designated as SLC6A4, protein fragments normally found in receptors for thrombin and HMGB1 such as thrombomodulin (THBD), protein fragments normally found in receptors for advanced glycation endproducts (AGEs) such the receptor for AGEs (RAGE), and protein fragments normally found in pattern recognition receptors (PRRs) such as soluble Toll-like receptor (sTLR2 and sTLR4).

In addition, the diagnostic and analytical systems, devices and methods disclosed herein can be expanded and enlarged, to enable additional biomolecular analyses that will indicate whether a person will tend to be more susceptible (compared to “normal” baseline levels) to long-term brain damage, or to post-concussion disorientation, depression, or similar problems, following a concussion, such as with pPCS leading to CTE, AD, PD, ALS or other serious consequences of undiagnosed repetitive mild TBI or concussions or even sub-concussive events. If someone is found to be especially susceptible to that type of damage or those types of problems, such as with genetic mutations of proteins involved in recovery from mTBI or concussion, they can be advised of that fact, and it can be taken into account in active plans such as by choosing certain sports and/or positions to engage in, while avoiding other types of sports or activities.

addition to providing diagnostic systems for early reading and evaluating brain injuries, in another aspect, applicant provides a method of treatment in tandem with these diagnostic systems. Applicant refers to these diagnostics systems and methods as “companion” to the therapeutic, which together form a “theranostic” combination.

As to the therapeutic aspects of the invention, medicaments, compounds and methods are provided for treating patients to help reduce and control unwanted neuroinflammation, and to help patients recover brain function after a neurological trauma or crisis, such as a head or spinal injury, a stroke or cardiac arrest, or a near-drowning or suffocation. These medicaments and compounds include chimeric proteins which combine: (i) a first polypeptide sequence derived from the “Box A” domain of the “High-Mobility Group Box 1” Protein ( HMGB1) protein; and, (ii) a second polypeptide sequence derived from the D1 lectin-like domain of thrombomodulin (TM).

These chimeric proteins (referred to herein as HMGB1/TM proteins) have combined-action effects that can both: (i) activate and promote certain repair-type functions within the brain, up to (and in some cases including) the growth of new neuronal fibers and/or the creation of new synaptic junctions; and, (ii) keep those types of inflammation-triggered repair processes within healthy and desirable limits.

Rather than being just single-purpose anti-inflammatory agents, the chimeric proteins of this invention trigger, tolerate, cooperate with, and support normal and healthy inflammatory responses, of the types, intensities, and durations that are associated with normal and healthy responses to injuries or infections. Subsequently, when an initial inflammatory response has finished doing its job, and should subside and become less active so that other cell types can drive and implement the subsequent stages of a healing and rebuilding process, these chimeric proteins can shift into their anti-inflammatory mode.

Immediately after a crisis, such chimeric proteins can be directly injected into brain tissues, such as into the vesicles where cerebrospinal tissues accumulate, or using advanced types of injection cannula that have been developed recently. Alternately or additionally, viral vectors which carry genes that encode such chimeric proteins can be transfected into certain types of neurons which have neuronal fibers that pass through the BBB and have accessible tips, such as olfactory receptor neurons, which have active and accessible neuronal fiber tips that can be directly contacted by viral vectors carried by nasal sprays.

In addition, new compositions of matter are disclosed herein, comprising modified (engineered) genes (as well as plasmids, non-pathogenic viruses, or similar genetic vectors which carry such genes in easily-handled and reproducible form) which encode the HMGB1/TM chimeric proteins described above, wherein the engineered genes have been altered to increase and enhance production of these chimeric proteins in specific selected types of host cells. For example, using known criteria, specialized computer programs have been and can be used to create modified codon selections that will encode the exact same amino acid residue sequence, wherein the codon selections will optimize the expression and production of these chimeric proteins in a specific type or class of host cell (such as human cells, insect cells, or yeast cells) which have been selected by a manufacturer of these proteins.

Furthermore, new compositions of matter also are disclosed herein, comprising chimeric proteins containing domains from both the HMGB1 and TM proteins, which have been modified by amino acid substitutions. Such modified sequences can be referred to as “tweaked”, engineered, or enhanced protein sequences, referred to as “muteins”. Such modified proteins can have certain advantages over corresponding “native” sequences. For example, cysteine residues which are not involved in creating disulfide bonds (which are “bridging”-type bonds between two cysteine residues, which help establish and stabilize the three-dimensional shapes and conformations of proteins) can be replaced by other amino acid residues, to avoid or minimize unwanted disulfide bonds which can alter the shape of a resulting protein in undesired ways. In addition, genes (and genetic vectors carrying such genes), which encode these mutein sequences also are within the scope of the invention.

In particular, as further detailed below, in additional aspects, a genetic vector containing an engineered gene sequence which encodes an initial polypeptide which contains at least one soluble domain derived from human thrombomodulin, at least one domain derived from the “Box A” domain of the “High-Mobility Protein Group, Box 1” ( HMGB1) human protein; and, a secretion sequence which will cause the at least a portion of the initial polypeptide containing the thrombomodulin and HMGB1 domains to be secreted by host cells which have synthesized the initial polypeptide. In this aspect, the engineered gene sequence can contain a plurality of substituted codons which have been selected and inserted into the engineered gene sequence to maximize expression of the initial polypeptide by a selected type of host cell, wherein the substituted codons do not occur in natural human genes which encode said thrombomodulin or HMGB1 domains, but are capable of encoding the same amino acids as the codons in natural human genes.

In another aspect, the genetic vector as described above, in an embodiment, is designed to transfect and replicate in human cells and is configured to express the initial polypeptide in a manner which causes the initial polypeptide to be processed and secreted by human cells in a smaller form which does not contain the secretion sequence. Also included within the scope of the invention is a genetic vector as described above, wherein the vector encodes an engineered protein from which one or more cysteine residues, which are not involved in disulfide bond formation, have been replaced by other amino acid residues.

In yet another aspect, a liquid preparation suited for intravenous injection into animals or patients or other medicaments containing a soluble chimeric protein is provided which contains at least one soluble domain derived from human thrombomodulin; and, at least one domain derived from the “Box A” domain of the “High-Mobility Group Box 1” Protein ( HMGB1) human protein, wherein the soluble chimeric protein is therapeutically useful for modulating neuro-inflammation.

In a further aspect, the liquid preparation or other medicament as described is provided which includes one or more cysteine residues which are not involved in disulfide bond formation, in native versions of said thrombomodulin and HMGB1 domains, having been replaced by other amino acid residues.

### BRIEF DESCRIPTION OF THE DRAWINGS

FIG. 1 is a schematic of the reader/device system comprising the device (300) interacting via Bluetooth with the smartphone (200), interacting via the Internet with central server (100) and the biomarker panel modules (30, 32).

FIG. 2 is a graphic showing plasma levels of HMGB1 in normal male control and males suffering an acute myocardial infarction (AMI), to establish normal levels of <4 ng/mL.

FIG. 3a is a graphic showing plasma levels of HMGB1 in TBI patients at day 1, day 3, day 30 and day 90 after brain injury; FIG. 3b shows HMGB1 plasma levels is essentially an acute occurrence since 48% of all TBI patients had elevated levels at day 1; FIG. 3c shows that elevated HMGB1 levels are independent of plasma tau and GFAP levels in the same TBI patients, indicating they identify a separate endophenotype of TBI.

FIG. 4 is a graphic showing plasma levels of another “mitochondrial releasate”, transcription factor of mitochondria A (TFAM), an HMG protein similar to HMGB1, in mice subjected to mild contusion TBI (CCI) compared to “sham” controls (no CCI) but under anesthesia and surgical manipulation, after 72 hours.

FIG. 5 is a graphic of the same samples subjected to immunoprecipitation (IP) either to polyclonal rabbit antibody to TFAM or control (“no treatment”) antisera and subjected to immuno-blot.

FIG. 6 is a graphic of relative amounts of mtDNA in plasma of mice subjected to CCI and quantified by qRT-PCR indicating maximum amounts at 24 hours post-TBI but remaining elevated even one month post-TBI. Note that mtDNA circulates in control mouse plasma in these experiments.

FIG. 7 is a schematic depiction of the domains of the gene and polypeptide sequences of an embodiment of the chimeric HMGB1/TM protein created as described herein. This schematic depiction accompanies and supports the complete sequence data, as contained in the Patentln sequence which accompanies this application.

### DETAILED DESCRIPTION OF THE INVENTION

As summarized above, this invention involves blood and fluid reader systems, materials, devices, and methods for evaluating the severity of traumatic head injuries, including concussions and other conditions, and thus, providing for removal of a player from the field, a trip to the hospital, a Medevac removal from the battlefield, or other steps to minimize brain damage. It further involves medicaments and methods of treatment of neuroinflammation involving chimeric compounds as disclosed herein. These systems, materials, devices, and methods are designed, in particular, for sites and situations where a relatively rapid determination must be made as to whether an athlete, soldier, accident victim, or anyone else who has been “stunned” or “shaken up”, by a blow to the head, a nearby explosion, or other comparable event, can rest for a brief period, “walk it off”, and then return to normal activity (such as to an ongoing athletic event, military patrol, etc.), or whether the person who suffered the blow to the head should receive prompt medical attention, to avoid or minimize lasting neurologic damage.

Accordingly, while the range and variety of uses for these types of diagnostic and evaluative materials, devices, and methods are not specifically limited to any particular class of events or activities, they should be regarded as being well-suited for use at events or in situations such as:

(1) athletic contests in which high-speed collisions between players occur, either as a normal part of the game (such as in boxing, extreme sports, football, hockey, etc.), or as less-frequent or incidental occurrences (such as in soccer, basketball, baseball, water polo, bicycle racing, etc.);

(2) during military activities, such as training exercises or patrols where accidents, ambushes, explosions, and similar events can occur;

(3) when “first responders” (which generally includes personnel who have been trained to respond to emergencies, such as police, firemen, ambulance attendants, etc.) arrive at the site of an accident or other incident or situation involving an apparent victim of a blow to the head or comparable trauma.

These systems, methods and materials can utilize diagnostic bioreagents that are affixed to surfaces of computer-readable devices, which are designed to be analyzed by electronic machines referred to herein as readers. The types of devices used for these analyses are classified as either:

(1) discs, if they spin while being “read” (usually by a laser beam); or,

(2) arrays, if they do not spin while being read.

Such devices are available commercially, including from one or more of Affymetrix, Illumina, GE Healthcare, Applied Biosystems, Beckman Coulter, Eppendorf Biochip Systems, Agilent, Claros/OPKO Health, SynVivo/CFDRC and Qloudlab and their use is known to those of skill in the diagnostic art and can be targeted to analyze the quantity or concentration of a “mitochondrial releasate” as described herein.

A “mitochondrial releasate” is firstly, a molecule (protein or nucleic acid) that is released from damaged or exploded mitochondria where it is, secondly, concentrated prior to its release. It might “originate” in the mitochondria or be transported there from the cell nucleus or cytoplasm, but is present and concentrated (about 5× or higher than that circulating in the blood) in the mitochondria prior to its release.

The form and suffix of the word “releasate” is intended to be similar to other words that relate to or involve fluid behavior, including “condensates”, “leachates”, “absorbates”, etc. Accordingly, to qualify as a mitochondrial releasate, a mitochondrial molecule may, in certain circumstances, also be defined as one that is released by injured, damaged, and/or dying cells, into circulating blood, in quantities that enable those particular types of molecules to be used to analyze the extent and severity of cellular or tissue damage in response to a traumatic blow or similar injury.

Not being bound hereby, there are at least the following types of candidate “mitochondrial releasates,” or mitochondrial DAMPs that are considered within the scope of the invention as offering substantial utility for evaluating the severity of a traumatic brain injury. These candidates can be summarized as follows:

1. First Category: Mitochondrial DNA (mtDNA), as an Entirety

This category includes DNA sequences and segments that are specific to mitochondrial genes, as compared to nuclear genes (i.e., genes carried within the nucleus of a cell). The prefix “mt”, in “mtDNA”, specifically indicates that the DNA is of distinctly mitochondrial origin. Strands of mtDNA will bind, with affinity and specificity, to “complementary” DNA strands that have been affixed to the surface of a spinning disc or non-spinning array that is suited for processing and handling by the types of portable “reader” machines that are well-known and widely used for handling medical diagnostics and biochemical research. Accordingly, the types of methods and procedures that normally are used to perform “Southern blots”, which are standard and well-known types of tests that are done in biochemistry labs around the world, can be used to measure the levels of mtDNA in the blood of an athlete, soldier, accident victim, or other person of interest.

To optimize the utility of these types of tests, an athlete, soldier, or other person who is at elevated risk of a collision, attack, or other form of

TBI preferably should be tested, at the beginning of a season, before being deployed to a combat zone, or at a comparable suitable time, to determine both:

(i) a normal ratio between that person's mtDNA, and his/her nuclear DNA; and,

(ii) the quantity of mtDNA in that person's blood, under normal conditions.

If either (i) that ratio, or (ii) the concentrations of both mtDNA and nuclear DNA, in that person's blood, is found to be significantly altered, after a blow to the head or similar event, then the altered ratio and/or increased concentrations should be treated and regarded as a warning signal that the blow to the head may have created a concussion, and should be treated as a potentially serious neurologic problem that requires prompt medical attention.

Two specific mitochondrial genes for targeted analysis as described herein can be:

(1) the mitochondrial gene which encodes a protein called Cytochrome B oxidase, discussed below; and,

(2) the mitochondrial gene which encodes a protein called ATP synthase, subunit 6.

2. Second Category: Degraded Mitochondrial Polynucleotides

In addition to testing for unaltered or native mtDNA, Southern blots can also be used to test for fragments of mtDNA that have been broken into pieces. That type of breakage is accelerated, in mtDNA as compared to nuclear DNA, by oxygen radicals (also referred to as oxygen free radicals, oxidative radicals, and “radical oxygen species” (ROS), which are found in abnormally high quantities inside the mitochondria (this is due to the fact that the mitochondria are the “cellular furnaces” where glucose (a sugar molecule) is “burned” (i.e., oxidized) to release its stored energy). Degraded mtDNA can also be assessed in a microfluidic chip using digital droplet-PCR (dd-PCR)

3. Third Category: Cytochrome C Oxidase

Cytochrome c oxidase is an enzyme which participates in the formation of “cytochrome c”, the so-called “death messenger” molecule, mentioned in the Background section. This protein enzyme is encoded by a “nuclear gene” (i.e., genes located on the chromosomes inside the nuclei of mammalian cells). However, once formed, these enzyme molecules migrate to (or are actively transported), i.e., they “translocate” to the mitochondria, and are taken inside the mitochondria to a point where almost no cytochrome c oxidase molecules remain as free molecules in the cytoplasm of a cell. Accordingly, cytochrome c oxidase provides an example of an enzyme (protein) that is found inside mitochondria, and which does not normally otherwise exist in substantial quantities in cytoplasmic fluid, or in blood samples in healthy people.

Its presence and concentration can be detected by either:

(i) assays or procedures that use monoclonal antibodies which bind specifically to cytochrome c oxidase; or,

(ii) assays which provide a substrate that is acted upon by Cytochrome C oxidase's enzymatic activity.

4. Fourth Category: Cyclophilin D (CypD)

“Cyclophilin D” is another protein which is encoded by genes in the nuclei, but which actively migrates or is transported to mitochondria. Normally, it is affixed to mitochondrial membranes, as part of an organelle structure called the “mitochondrial permeability transition pore”. As such, it normally is located near the outer surfaces of the mitochondrial membranes, and it will be released, in a relatively rapid manner, if mitochondrial membranes in a cell are seriously damaged, to a point which causes them to rupture and break apart. It can be detected by assays that use monoclonal antibodies that bind specifically to cyclophilin D.

5. Fifth Category: Atp Synthase, Subunit 6

This protein, which is encoded by mitochondrial genes, normally is found at significant quantities, in healthy tissue, only in mitochondria, rather than in cytoplasm or blood. It can be detected by assays that use monoclonal antibodies that bind specifically to subunit 6 of the ATP synthase complex.

6. Sixth Category: High Mobility Group Box 1 Protein ( HMGB1)

This protein, also encoded by a gene in a cell's nucleus, translocates to the mitochondria where it affects “mitochondrial quality control” or mitophagy. It is a prominent pro-inflammatory mediator; and, as amphoterin, it has been shown to be essential for normal brain development. As in other traumatic injuries, it is increased in brain and spinal cord tissue after injury, and is released into the bloodstream.

7. Seventh Category: Transcription Factor for Mitochondria A (TFAM)

This protein transcription factor, also encoded by a nuclear gene, is responsible for mitochondrial biogenesis. TFAM is normally bound to and remains associated with mitochondrial DNA (mtDNA) when released from damaged cells. It is released from the brain after TBI. Like HMGB1, TFAM is a member of the high-mobility group ( HMG) of proteins because it contains two HMG “boxes”. These factors make TFAM a promising “analyte” for use as described herein.

Other Biomolecules that can Indicate Increased Susceptibility to Long-Term Problems After One or More Concussions

In addition to the types of candidate molecules listed above, any of various additional types of bioreagents (such as monoclonal antibodies or strands of DNA which have specific binding affinity for any targeted biomolecules of interest in a person's blood) can be affixed to a diagnostic disc, array, or other device as described herein, to assist coaches, trainers, physicians, and others determine a certain person's susceptibility to neurodegenerative decay, and/or to mental or behavioral problems (such as lingering depression, disorientation, etc.), following a concussion or other head trauma.

Accordingly, if a blood test is undertaken on a candidate athlete, either at the start of a season or at an important milestone in his or her career (such as when a candidate athlete is applying for an athletic scholarship to college, or is attempting to be selected for a professional sports team), it is considered within the scope of the invention to conduct a blood test and to use that same blood test to check for other, additional factors that may indicate a greater-than-normal susceptibility to long-term neurological, mental, or behavioral problems, if that candidate suffers a concussion.

The following molecules are believed to be candidates for testing and analysis to determine higher-than-baseline levels of susceptibility to long-term neurological problems, following a concussion.

Accordingly, the types of diagnostic discs and arrays disclosed herein, or otherwise known to those skilled in this art, can be designed to include reagents that will provide physicians, coaches, trainers, and other analysts with data that can indicate the blood-borne concentrations of any or all of the following biomolecules:

(1) Apolipoprotein E (APO-E);

(2) TAR DNA binding protein (TDP-43; TARDBP);

(3) Angiotensin-converting enzyme (ACE); other “serpin” proteins, such as protease nexin 1 (PN1; SERPINE1); neuroserpin (SERPINI1); and, plasminogen activator inhibitor 1 (PAI-1; Serpinel or SERPINE1);

(4) cytokine genes and inflammatory mediators, and/or protein subunits from their receptors; examples include Interleukin-1 beta (abbreviated as IL-1β or IL-1B), and tumor necrosis factor alpha (abbreviated as TNF-α, TNF-A, or simply TN F);

(5) Thrombomodulin (THBD), and endothelial cell protein C receptor (EPCR; PROCR); soluble forms of both (i.e, sTM and sEPCR);

(6) 5-hydroxytryptamine (serotonin) receptor 2A (5-HT2A; HTR2A) and solute carrier family 6 (neurotransmitter transporter, 5-HT) and/or member 4 (SLC6A4; or SERT);

(7) Certain types of “high mobility group” proteins, including “HMG box 1 protein” ( HMGB1); and,

(8) certain proteins referred to as “RAGE” proteins (which refers to “receptor for advanced glycation endproducts”, also given the acronym AGER), soluble form (i.e., sRAGE).

As an example of the diagnostic blood reader system 100 of the invention, interfacing with a portable computer, smartphone or tablet 200, a portable handheld reader device 300 with individual protein or DNA detecting modules 30 and a stylet 40 for inducing a pinprick drop of blood (FIG. 1) is shown: In this depiction a reader 300 sold by Qloudlab, the Sceptre® device is used, but others are commercially available and known to those skilled in blood and other fluid analysis. This contains click-out modules 30 each with a separate sub-panel of the “mitochondria! releasates” 32 and click-out stylet 40 to prevent inter-patient contamination.

In FIG. 2 levels of the DAMP (mtDAMP) HMGB1 in normal aged-matched controls (mean about 1.5) is compared with myocardial infarct patients (mean of almost 15). From the world literature normal blood values for this DAMP do not exceed 4 ng/mL.

### Studies in Humans:

HMGB1 is an example of a first “mitochondrial releasate”. It is a nucleus-encoded, non-histone nuclear protein that is translocated to mitochondria to effect mitochondrial quality control and mitophagy. In the example depicted, a highly sensitive and accurate HMGB1 ELISA Kit was used. Seventy-five days were analyzed (day; 44, day30 and 50 day90) banked repository plasma samples from mild-moderate TBI patients, for whom day 1 and then 3 subsequent post-injury samples were used. As shown in FIG. 2, HMGB1 levels are typically <4 ng/mL in control adult blood, whereas post-TBI (FIG. 3a), HMGB1 levels were markedly elevated (48%) within the first 24 hrs after TBI as shown in FIG. 3b.

A number were >10 times normal plasma levels seen in FIG. 1. They subsequently declined but a few remained elevated at day 90. In most instances HMGB1 concentrations declined down to or close to baseline normal values by 30 days after injury, but in a number of patients a 2^{nd} phase of elevated HMGB1 levels occurred at 90 days (FIG. 3a). The descriptive statistics for the plasma samples from post-TBI patients are shown in Table 1.

TABLE 1
Descriptive statistics for TBI samples

| | N | Min | 1stQ | Median | Mean | 3rdQ | Max |
|---|---|---|---|---|---|---|---|
| HMGB1 | | | | | | | |
| d 1 | 75 | 2.5 | 2.5 | 3.04 | 7.921 | 5.95 | 71.7 |
| d 3 | 31 | 2.5 | 2.5 | 2.69 | 4.659 | 4.425 | 21.1 |
| d 30 | 44 | 2.5 | 2.5 | 2.5 | 2.96 | 2.5 | 11.07 |
| d 90 | 50 | 2.5 | 2.5 | 2.5 | 3.412 | 2.5 | 26.44 |
| GFAP | | | | | | | |
| d 1 | 31 | 0 | 3.795 | 15.9 | 85.843 | 83.9 | 863.3 |
| d 30 | 32 | 0 | 0.7415 | 1.325 | 1.8409 | 2.02 | 10.42 |
| d 90 | 23 | 0.282 | 0.894 | 1.35 | 1.919 | 2.195 | 6.43 |
| Tan | | | | | | | |
| d 1 | 32 | 2.9 | 6.103 | 9.56 | 18.288 | 16.3 | 85.5 |
| d 30 | 32 | 2.9 | 4.755 | 6.665 | 10.678 | 9.047 | 97.8 |
| d 90 | 23 | 2.18 | 3.94 | 5.72 | 6.007 | 6.97 | 15 |

Also compared were the plasma concentrations of tau and GFAP proteins in the same study samples to the current HMGB1 values (Table 1). Concentrations of these proteins, tau for neuronal and GFAP astrocytic, respectively, required use of single-molecule technology (SIMOA, Quanterix) since they are in the pg/mL range. In contrast, HMGB1 bloodstream concentrations are 1000-fold greater, in the ng/mL range, and are read easily by a conventional bench-top or even point-of-care (POC) ELISA reader. These are graphically depicted in FIG. 3c. In addition to dayl, day30 and day90, day3 was also analyzed for HMGB1 levels. Also measured were levels of HMGB1 in plasma from another cohort of TBI patients. Similar findings were found.

### Studies in Rodents:

Example of the second mitochondrial “releasate” as part of a test set of the invention to be used in blood reader system, (see claim 1) is transcription factor of mitochondria A (TFAM), an HMG protein similar to HMGB1. TFAM is encoded by a nuclear gene and translocated to the mitochondria, as is HMGB1, with which it shares structural and functional homologies.

In intact mitochondria TFAM is normally bound tightly to mitochondrial DNA (mtDNA), and this is also true for it when it is released from exploding necrotic cells and disrupted mitochondria. Together with mtDNA, bound TFAM is termed “nucleoids.” This example comes from controlled cortical contusion (CCI) model of mild TBI in mice. Blood was drawn at 4, 8, 24 and 72 hours after CCI and subjected to Western blot (insert) and then ELISA with results calculated based on relative levels, shown in FIG. 4, and compared to sham control. To show specificity for TFAM, anti-TFAM polyclonal antibody (rabbit) was used to immunoprecipitate (IP) mouse TFAM from post-TBI blood. This is shown in FIG. 5, alongside IP with non-specific antisera.

Similar results were obtained with repetitive concussion model in mice (not shown), and in this study involving retrospective human TBI samples.

As an example of a third “mitochondrial releasate” as part of a test set (see initial claim 2) is native mtDNA, as shown in FIG. 6. This study was performed in young adult male mice and used CCI to induce mTBI.

Thus, there has been shown and described a new and useful set of diagnostic blood and fluid reader systems, devices and methods which can help coaches, trainers, first-responders, military personnel, physicians, and others, in their efforts to evaluate the severity of damage that may have been caused by a concussion or other head trauma. Although this invention has been exemplified for purposes of illustration and description by reference to certain specific embodiments, such as brain injury or TBI, it will be apparent to those skilled in the art that various modifications, alterations, and equivalents of the illustrated examples are possible. For example, new and useful sets of diagnostic systems as outlined by the Applicant, may be used to identify those individuals having had a brain injury who may ultimately progress to neurodegenerative diseases such as AD, PD, ALS, CTE or others. Any such changes which derive directly from the teachings herein, and which do not depart from the spirit and scope of the invention, are deemed to be covered by this invention.

As also briefly summarized above, this invention also discloses medicaments, compounds, medical uses and methods of treatment relating to neuroinflammation for conditions such as mTBI, for chimeric proteins which combine:
(i) a polypeptide sequence derived from at least one soluble domain of thrombomodulin (which normally is a membrane-bound immobilized protein); with,
(ii)a second polypeptide sequence derived from the “Box A” domain of the “High-Mobility Group Box 1” Protein ( HMGB1) protein.

These chimeric proteins have a double-action effect that renders them useful for helping to reduce and control neuroinflammation (defined herein to be limited to inflammation that occurs within CNS tissue in the brain or spinal cord, which includes the membranes that surround the brain and spinal cord). Administration of these protein molecules (via intravenous injection, an implanted infusion pump, genetic vectors, or similar means) can help control and reduce brain or spinal damage following an acute crisis such as a stroke, traumatic injury to the head or spine, etc.

Alternately, these chimeric proteins can help reduce and control chronic and/or low-level neuroinflammation, as occurs in various types of progressive neurodegenerative diseases and other disorders.

Samples of these chimeric proteins have been created, for testing purposes, using known and published gene and protein sequences from the human versions of both proteins (rather than from corresponding genes or proteins in mice, rat, or other animals) for both “source proteins” (i.e., thrombomodulin, and HMGB1). The selected gene domains (which also can be called gene fragments, partial sequences, etc.) from the TM and HMGB1 proteins were assembled as depicted in FIG. 7, which displays both the DNA codons and the corresponding amino acids, aligned with each other in FIG. 7. The “native” gene sequences were modified (in ways that can be referred to as engineered, enhanced, “tweaked”, or similar terms) to give both:
(i) modified gene sequences (i.e., the sequence of nucleotide bases in the DNA which forms the gene, which was created in the form of a plasmid which was designed for optimal expression levels in human cells); and,
(ii) an amino acid sequence that was modified somewhat, in the chimeric proteins expressed by the engineered genes, as described below (such as by adding a “histidine tail” to the carboxy end of the amino acid sequence).

The modification, engineering, or “tweaking” of the codons arises from several factors that have become known and appreciated by genetic engineers, which include the following:
(1) There is a substantial amount of duplication among the codons, in the classic “genetic code”. There are 64 possible “codons” (this arises from the fact that each “codon” contains 3 bases, with 4 possibilities for each base (represented by A=adenosine, T=thymidine, C=cytosine, and G=guanidine in DNA, where T=thymidine is replaced by U=uracil in messenger RNA). Since 4×4×4=64, there are 64 possible codons. However, those 64 codons encode only 20 “primary” amino acids, in all proteins. As one example, six different codons (which are CGU, CGC, CGA, CGG, AGA, and AGG) will cause a residue of arginine (one of the 20 primary amino acids) to be added to a growing polypeptide chain, when a strand of protein is being “translated” from messenger RNA.

Over the course of evolution, different classes of organisms developed different “preferences” for which particular codons a certain type of “host cell” will use most frequently, and least frequently, in its genes. This enables a very useful form of control over the quantities of proteins that are expressed, by cells in different bacteria or organisms. If a certain protein is needed in large quantities, then the genes which encode that protein will tend to contain codons which are preferred, by that class of organism; conversely, if the quantity of a certain protein needs to be limited and somewhat suppressed, the genes which encode that protein will tend to contain codons which are non-preferred by that type of organism.

Through statistical analysis, biochemists have determined which are the preferred and non-preferred codons for each of the major “candidate” classes of host cells that can be grown in suspension cultures and used to synthesize desired proteins, via plasmids or other genetic constructs. Accordingly, the lists of preferred and non-preferred codons for the different types of cells that are commonly used to synthesize proteins via gene expression are all known. These generally range from E. coli, which are bacteria cells at the “cheapest and most prolific” end of the scale, up to human cells at the “most expensive and slowest-growing” end of the scale, with yeast cells and insect cells providing two “mid-point” levels between the cheapest and costliest ends of that spectrum.

All four of those cell types (i.e., bacterial, yeast, insect, and human) will generate exactly the same polypeptide sequences, when using the same starting genes, since the genetic code is exactly the same for all of those cell types; however, there are important differences in how each of those host cell types will handle several processes that fall within the label, “post-translation processing”, when specific types of proteins are being formed. The four most important types of “post-translation processing” involve:
(1) “folding” of a polypeptide strand, as it is being created by translation of an mRNA strand, into either a “desired and optimal” three-dimensional shape (which is usually fairly predictable, if human cells are used) or a “less desired and non-optimal” shape (which poses a greater risk when non-human host cells are used). The three-dimensional molecular shapes that are created by the folding processes that occur, as protein strands are assembled from amino acids by ribosomes within a cell, are usually referred to as protein “conformations” in the literature;
(2) creation of di-sulfide bonds, which are formed if two residues of the amino acid cysteine are positioned close enough to each other to enable their reactive “sulfhydryl” groups (R—SH) to jettison their hydrogen atoms and form an R—S—S—R link between two different cysteine residues at different locations in the polypeptide chain. Disulfide bonds are used to stabilize the three-dimensional shapes of protein molecules, and when they occur naturally in a protein, they usually are essential for proper functioning of the protein;
(3) creation of bonds involving atoms of zinc, iron, or other mineral elements (or, in some cases, organic “co-factors”) which also are used to establish and stabilize the desired three-dimensional shapes of proteins; and,
(4) “glycosylation”, which refers to enzyme-controlled addition of sugar groups to the accessible surfaces of a protein molecule.

If a problem occurs in any of those four types of post-translation processing, it can reduce (and in some cases destroy) the desired activity of a protein which is being synthesized by a chosen type of host cell; and, the likelihood of such problems increases, as candidate host cells move farther away from the human species. The per-unit costs of protein synthesis will decrease, in a step-wise fashion, as one moves “downward” in terms of host cell complexity (i.e., from human cells, to insect cells, to yeast cells, to bacterial cells); however, the likelihood of “post-translation processing” problems increases, along each step of that pathway. Therefore, when trying to establish and confirm the utility of a new type of chimeric protein, most research teams that work in this field will initially use human cells as the host cells for synthesizing their new protein; then, once the safety and efficacy of the new protein has been shown by reliable and reproducible results, the researchers can then begin to evaluate which type of host cell will provide the best balance of high efficacy and safety, versus lower manufacturing costs.

There also are several other factors, which must be taken into account, when a gene is being optimized for protein production in a chosen type of host cell. For example:
(i) “over-abundant” or deficit levels of C (cytosine) and G (guanosine) residues should be avoided; in general, the frequency of C and G residues, in any large section of a gene, should be kept at a level higher than about 30%, and less than about 80%;
(i) sequences of about 10 or more bases that have unusual clusters of either A and T bases, or G and C bases, also should be avoided;
(ii) certain types of sequences or structures, known among genetic engineers as “TATA boxes”, “chi-sites”, and “ribosomal entry sites”, also should be avoided, unless they have been recognized and are specifically desired at certain sites in a gene;
(iii) repeating sequences, and “complementary” sequences that could cause a single-stranded mRNA molecule to form a loop and create a segment of double-stranded mRNA (where the RNA bases would bind to each other in a stable manner, comparable to the type of attraction that normally holds together double-stranded DNA) should be avoided; and,
(iv) types of sequences that have been identified by genetic engineers as “splice donor sites” or “splice acceptor sites”, in cells from eukaryotic animals, also should be avoided.

After a researcher has settled upon the exact sequence of amino acids which will be present in a desired type of protein (either chimeric or otherwise) that will be synthesized by host cells, and also has selected the type of host cells that will be used to synthesize the protein, the “native” gene sequences can be analyzed by a computer program which takes all of the factors mentioned above into account, and which will determine an optimized gene sequence for synthesis of the desired protein, by the selected type of host cells. There are several service companies that provide those types of computerized analyses, for researchers and small companies that want to create batches of chimeric or other proteins via cellular fermentation. The Applicant herein used the services of one of those companies (LifeTechnologies, a division of Thermo Fisher Scientific Inc., with a website at lifetechnologies.com), which developed a software program called GeneOptimizer(™) to perform the types of analyses described above.

Accordingly, the sequence data disclosed herein show the computer-optimized gene sequences. Those sequences are presented as “sense strand DNA” sequences, which show the sequence that resembles and corresponds to the messenger RNA strand that will be expressed by the gene, with T (thymidine) residues (which appear in DNA) in place of the U (uracil) residues that appear in mRNA. As is conventional, the DNA sequence progresses from the so-called 3′ end, toward the 5′ end; and, the amino acid sequence progresses from amino end of the protein, toward the carboxy end.

Turning again to FIG. 7, a schematic is depicted that indicates the various different domains of the gene and polypeptide sequences of the chimeric HMGB1/TM protein that was created as described herein. This schematic depiction accompanies and supports the complete sequence data, which is contained in the Patentln sequence, which accompanies this application.

The domains shown in FIG. 7 include the following:
(1) a “leader” sequence containing bases 1-38, which was present in the mRNA strand, but which was not “translated” into amino acids when the mRNA strand was processed by a ribosome; in any strand of mRNA, any bases which are positioned in front of the

ATG “start codon” (which establishes the “reading frame” for all of the codons which follow) is called a leader sequence;
(2) a “transit peptide” sequence of 60 bases in the DNA (i.e., bases 39 through 98, which contained 20 codons, starting with the ATG “start codon” in bases 39-41), which encoded 20 amino acid residues (including the initial methionine residue which became the amino terminus of the protein) of a well-known “secretion sequence” (also called a leader sequence, a transit or transport sequence, or similar terms), which caused the newly-formed protein molecules to be secreted into the extra-cellular liquid by the host cells, rather than allowing or causing the protein to accumulate in large quantities inside the host cells;
(3) a sequence of 234 bases in the DNA (i.e., extending from base 99 through base 332, containing 78 codons), which encoded 78 amino acid residues that contain the native sequence (including native cysteine residues) of the so-called “DNA binding domain” of the “High Mobility Group, Box 1” human protein;
(4) a sequence of 486 bases in the DNA (i.e., extending from base 333 through base 818, containing 162 codons) which encoded 162 amino acid residues that contain the native sequence (including native cysteine residues) of the “C-type lectin domain” of human thrombomodulin;
(5) a sequence of 18 bases in the DNA (i.e., extending from base 819 through base 836, containing 6 codons) in the DNA, which encoded six histidine residues, which were added to create a “histidine tail” at the carboxy end of the chimeric protein; and,

The “transit peptide” sequence mentioned above (containing 20 amino acid residues) was cleaved off of the mature protein, and was not part of the final extra-cellular protein, which contained 246 amino acid residues.

The addition of “histidine tails” to the carboxy ends of engineered proteins is a widely-used technique for enabling improved purification of such proteins, since histidine tails will bind to beads that have been loaded into an “affinity column” if the beads contain nickel (i.e., the metal/mineral element, abbreviated as Ni). Proteins with “histidine tails” will strongly and tightly bind to nickel-containing starch beads, under the initial set of conditions that are used to pass a protein suspension through an affinity column, while essentially everything else in the liquid suspension will pass through the column and can be discarded. After the liquid containing the unwanted proteins has finished passing through the affinity column, a different type of “elution buffer” liquid (usually with higher salt and/or acidity levels, or containing specialized “releasing” chemicals such as imidazole) is then passed through the affinity column, to release the proteins with the histidine tails from the nickel-containing beads. This allows the protein molecules of interest to exit the column, and be collected in relatively pure form, in the elution buffer. This is a standard technique in genetic engineering, and it was used to facilitate handling and purification of the chimeric proteins (with histidine tails) described herein; however, as described below, that type of purification did not result in the expected levels of purification, and an additional challenge arose which had to be overcome by the Inventor, as described below.

The engineered gene was synthesized, and inserted into a plasmid designed for such purposes, designated as the pcDNA3.1(+) A009 plasmid by LifeTechnologies. The plasmid vector contains Kpnl and EcoRl insertion sites, positioned behind a “strong mammalian promoter” (so-called “strong promoters”, often derived from genes carried by viruses, are used to cause abnormally large quantities of mRNA strands to be expressed by engineered genes). This allowed the synthesized fragment of double-stranded DNA to be inserted, in the desired orientation, directly behind the strong promoter in the plasmid. Any such inserted gene also can be removed and isolated from that plasmid, if desired, and inserted into any other selected genetic vector for transfection of any other type of host cell.

The resulting plasmid was used to transfect a line of human embryonic kidney (HEK) cells designated as the 293-F line of cells. These were created, by LifeTechnologies, from an earlier cell line called the HEK-293 cell line, which emerged in the 1990′s as a “standard workhorse” mammalian cell line used in numerous molecular biology labs around the world, with a large assortment of published articles describing how that cell line was created, and how the cells have been used to synthesize numerous different types of foreign proteins. Briefly, the HEK-293 cell line is a “transformed” or “immortal” cell line, which was transformed by inserting cancer-related “oncogenes” into the cells, in a manner that enables HEK-293 cells to reproduce indefinitely in suspension culture medium (that trait is not shared by non-transformed mammalian cell strains). That transformation, using a small and limited number of cancer genes, did not interfere with most of the normal metabolic processes of these particular cells. Accordingly, the 293 line of HEK cells became an immortal mammalian cell line which is used very widely, in biology labs around the world, to express chimeric, foreign, or otherwise engineered genes into proteins.

By using additional genetic engineering and cell-screening techniques, known to those skilled in the art, at Applicant's request, a company known as LifeTechnologies created a line of additionally-modified HEK-293 cells, designated the “FreeStyle 293” or “293-F” cell line. 293-F cells can be grown in “serum free” culture medium, which did not need to contain certain types of expensive reagents that are required by normal HEK-293 cells. Cells transfected with the chimeric-producing materials were cultured for 7.5 days in a two liter reactor. The cell suspension was then passed through a filter which allowed the extra-cellular liquid and secreted proteins to pass through, while retaining the cells.

The resulting liquid was divided into two halves, as a quality control check to make sure both portions would perform in the same way during subsequent processing. Each half was passed through a gel filtration chromatography column packed with HiLoad Superdex 75 16/60 (sold by GE Healthcare Life Sciences), and the aliquots that emerged were analyzed for absorption of ultraviolet radiation, which indicates protein concentrations. Distinctive humps indicating high concentrations of a specific protein appeared in aliquots 8 through 14 of one portion, and aliquots 9 through 15 of the other portion, so aliquots 9-15 from both runs were mixed together, and further purified using gel electrophoresis. Small samples (0.09 ml each) were removed and tested for molecular weight, purity, and amino acid sequencing, while the remainder was frozen for storage and future use.

Molecular weight, sequencing, and purity testing confirmed that the denatured-then-refolded protein preparation had: (i) the expected molecular weight; (ii) the amino acid sequence which corresponded to the engineered gene construct shown in FIG. 7; and, (iii) sufficient purity for testing either in vitro (i.e., in cell culture tests), or in vivo (i.e., in lab animals).

As known by those skilled in the art, e.g., workable close sequence identity of alternative forward, reverse primers and probes, e.g., such as those within the range of 70%-99.9% sequence similarity, within 5 bps-50 bps of sequence length, and/or including additions and deletions to such sequences, and any modification made by sliding or shifting the sequences by a few nucleotides, or by other molecular manipulation known to those skilled in the art, which may be designed or discovered and tested, to provide comparable genes and proteins to the chimeric-protein producing genes and chimeric proteins of the present invention, given the teachings of this invention. Such workable alternative primers and probes, together with those that use any degenerate or alternative bases for making any of the primers and probes to produce the same, or other know or to-be-developed molecular constructs, are within the scope of the present invention.

The resulting preparation is a new composition of matter, designed by the Applicant to have a highly useful therapeutic ability to help modulate and control neuro-inflammatory episodes and conditions of the type that are described in the Background. These proteins can be administered to patients in need of such treatments, by any of various means known to neurologists. For example, to treat cases of acute and/or severeneuro-inflammation (such as during a recovery period following an injury, stroke, infection, etc.), these chimeric proteins can be injected into the extracellular liquids that contact and lubricate the membranes which surround the brain and spinal cord (such as by epidural or subarachnoid injections or infusions), or into the ventricles which handle cerebrospinal fluids inside the brain).

Alternately or additionally, they can be injected directly into brain tissue, using specialized micro-injection devices designed for brain or spinal tissue, such as described in US patent 5792110 (Cunningham 1998). To treat non-acute cases and/or cases which will require a sustained series of treatments over a span of weeks or months, they can be introduced into brain or spinal tissue that is protected by the BBB, by means of genetically engineered vectors that will be actively taken into the tips of neuronal fibers that cross the BBB and are accessible outside the section.

BBB (such as the tips of olfactory receptor neurons, as just one example), as described in published US patent applications 20030083299 and 20060280724 (both by Ferguson). It is understood that any other suitable administration method, either currently known or hereafter developed, also can be used if desired and is also within the scope of the invention.

### EXAMPLES

### Example 1

### Computer-Aided Design of the DNA Sequence

A chimeric HMGB1/TM protein of the type and description shown in FIG. 7, was synthesized at Applicant's direction by LifeTechnologies (a division of Thermo Fisher Scientific Inc., with a website at lifetechnologies.com) to optimize the DNA sequence that was used to create that polypeptide, via fermentation of a genetically-transformed human-derived cell line using a gene sequence optimizer program.

### Example 2

### Synthesis of the HMGB1/TM Chimeric Protein

An engineered gene having the DNA sequence shown in FIG. 7 was synthesized (using automated equipment) at LifeTechnologies. It was inserted into a plasmid designated as the pcDNA3.1(+) A009 plasmid, which contains Kpnl and EcoRl insertion sites positioned behind a strong mammalian promoter. The use of both types of unique restriction sites, in that plasmid, ensured that the synthesized DNA fragment was inserted into the plasmid in the desired orientation, in a location where high levels of mRNA transcription would be driven by the strong gene promoter. The plasmid construct was replicated in E. coli cells, and the copies were used to transfect a human-derived transformed (immortal) cell line designated as “293-F” cells. These cells were initially obtained from “human embryonic kidney” (HEK) tissue, and arose from a cell line called HEK-293 cells, which are a well-known “standard workhorse” mammalian cell line used in numerous molecular biology labs around the world.

By using additional genetic engineering and cell-screening techniques, a line of further-modified HEK-293 cells, called the “FreeStyle 293” or “293-F” cell line was created. Unlike the parent line of HEK-293 cells, 293-F cells can be grown in “serum free” culture medium that does not need to contain certain types of expensive nutrient-type reagents, such as mammalian blood serum. Applicant's HMBG1/TM plasmid was transfected inside such 293-F cells using standard techniques.

The transfected cells were cultured for 7.5 days in a two liter reactor, in “FreeStyle 293 Expression Medium”. The cell suspension was then passed through a filter, which allowed extra-cellular liquid and secreted proteins to pass through the filter, while the cells were retained on the filter surface.

Example 3

### Purification of Denatured Protein

HMGB1/TM protein was purified by denaturing the protein, by using a buffering liquid which disrupts and relaxes the three-dimensional shape of the protein without changing or altering its amino acid sequence. That denaturing buffer contained a high level of salt, along with dithiothreitol (DTT), urea, and trimethyl-glycine (commonly known as betaine).

When mixed with that solution, the chimeric protein bound to the nickel-containing starch in an affinity column, in a selective manner which allowed the protein to be purified. It was released from the nickel-containing starch in the column (by using an elution buffer containing imidazole) so that it could exit the column and be recovered. It was then dialyzed into a urea buffer, which allowed it to refold back into its native configuration.

The resulting liquid was then passed through a gel filtration column loaded with HiLoad Superdex 75 16/60, using a buffer that contained 20 mM

HEPES, 200 mM NaCl, 1 mM dithiothreitol, 1 mM EDTA, 50 mM betaine, and 10% glycerol (pH 7.4). When tested, using gel electrophoresis, which separates biomolecules based on size and charge, in a manner which effectively removed the denaturing reagents from the proteins of interest. The gel material used during the electrophoresis was dissolved and removed, which allowed the protein (suspended in a liquid buffer) to refold, which presumably returned the large majority of the protein molecules into their normal conformation, using the same non-eluting buffer liquid that had been used for the prior affinity column passaging.

The resulting liquid was divided into two halves, as a quality control check to make sure both portions would perform in the same way during subsequent processing. Each half was passed through a gel filtration chromatography column packed with HiLoad Superdex 75 16/60, using a buffer that contained 20 mM HEPES, 200 mM NaCl, 1 mM dithiothreitol, 1 mM EDTA, 50 mM betaine, and 10% glycerol (pH 7.4). The aliquots that emerged were analyzed for absorption of ultraviolet radiation, which indicates protein concentrations.

Distinctive humps indicating high concentrations of a specific protein appeared in aliquots 8 through 14 of one portion, and aliquots 9 through 15 of the other portion, so aliquots 9-15 from both runs were mixed together, and further purified using gel electrophoresis. Small samples (0.09 ml each) were removed and tested for molecular weight, purity, and amino acid sequencing, while the remainder was frozen for storage and future use.

Molecular weight, sequencing, and purity testing confirmed that the refolded protein preparation had: (i) the expected molecular weight; (ii) the amino acid sequence which corresponded to the engineered gene construct shown in FIG. 7; and, (iii) sufficient purity for testing either in vitro (i.e., in cell culture tests), or in vivo (i.e., in lab animals).

Thus, there has been shown and described a new and useful means for creating both (i) a new type of chimeric protein with a highly beneficial medical utility, and (ii) an improved and enhanced gene construct which can be used to manufacture that chimeric protein, by using fermentation via human or other eukaryotic cells. Although this invention has been exemplified for purposes of illustration and description by reference to certain specific embodiments, it will be apparent to those skilled in the art that various modifications, alterations, and equivalents of the described examples are possible. Any such changes which derive directly from the teachings herein, and which do not depart from the spirit and scope of the invention, are deemed to be covered by this invention.

PATENTIN SEQUENCE

NON-TRANSLATED:
CCGCGCTGGTTACTTAAGCTTGGTACCGCCACC //
Secretion Signal, 20 CODONS
ATGGAGACCGACACCCTGCTGCTGTGGGTGCTGCTGCTGTGGGTGCCCGGCAGCACCGGC
M E T D T L L L W V L L L W V P G S T G
MetGluThrAspThrLeuLeuLeuTrpValLeuLeuLeuTrpValProGlySerThrGly
CODONS/RESIDUES 21-98 = High-mobility Group Protein B1
GGCAAGGGCGACCCCAAGAAACCCAGAGGCAAGATGAGCAGCTACGCCTTCTTTGTGCAG
G K G D P K K P R G K M S S Y A F F V Q
GlyLysGlyAspProLysLysProArgGlyLysMetSerSerTyrAlaPhePheValGln
ACCTGCAGAGAGGAACACAAGAAGAAGCACCCCGACGCCAGCGTGAACTTCAGCGAGTTC
T C R E E H K K K H P D A S V N F S E F
ThrCysArgGluGluHisLysLysLysHisProAspAlaSerValAsnPheSerGluPhe
AGCAAGAAATGCAGCGAGCGGTGGAAAACCATGAGCGCCAAAGAGAAGGGCAAGTTCGAG
S K K C S E R W K T M S A K E K G K F E
SerLysLysCysSerGluArgTrpLysThrMetSerAlaLysGluLysGlyLysPheGlu
GACATGGCCAAGGCCGACAAGGCCAGATACGAGAGAGAGATGAAGACCTATATCGCCCCT
D M A K A D K R Y E R E M K T Y T Y I A P
AspMetAlaLysAlaAspLysAlaArgTyrGluArgGluMetLysThrTyrIle // AlaPro
GCCGAGCCTCAGCCTGGCGGCTCTCAGTGCGTGGAACACGACTGCTTCGCCCTGTACCCC
A E P Q P G G S Q C V E H D C F A L Y P
AlaGluProGlnProGlyGlySerGlnCysValGluHisAspCysPheAlaLeuTyrPro
GGACCCGCCACCTTCCTGAACGCCAGCCAGATCTGCGACGGCCTGCGGGGCCATCTGATG
G P A T F L N A S Q I C D G L R G H L M
GlyProAlaThrPheLeuAsnAlaSerGlnIleCysAspGlyLeuArgGlyHisLeuMet
ACCGTGCGGTCTAGCGTGGCCGCCGACGTGATCAGCCTGCTGCTGAATGGCGACGGCGGC
T V R S S V A A D V I S L L L N G D G G
ThrValArgSerSerValAlaAlaAspValIleSerLeuLeuLeuAsnGlyAspGlyGly
GTGGGACGGCGGAGACTGTGGATTGGACTGCAGCTGCCCCCTGGCTGCGGCGACCCTAAG
V G R R R L W I G L Q L P P G C G D P K
ValGlyArgArgArgLeuTrpIleGlyLeuGlnLeuProProGlyCysGlyAspProLys
AGACTGGGCCCCCTGAGAGGCTTCCAGTGGGTGACAGGCGACAACAACACCAGCTACAGC
R L G P L R G F Q W V T G D N N T S Y S
ArgLeuGlyProLeuArgGlyPheGlnTrpValThrGlyAspAsnAsnThrSerTyrSer
AGATGGGCCAGACTGGACCTGAATGGCGCCCCTCTGTGCGGCCCTCTGTGTGTGGCTGTG
R W A R L D L N G A P L C G P L C V A V
ArgTrpAlaArgLeuAspLeuAsnGlyAlaProLeuCysGlyProLeuCysValAlaVal
TCTGCCGCCGAGGCCACCGTGCCTAGCGAGCCCATTTGGGAGGAACAGCAGTGCGAAGTG
S A A E A T V P S E P I W E E Q Q C E V
SerAlaAlaGluAlaThrValProSerGluProIleTrpGluGluGlnGlnCysGluVal
AAGGCCGACGGCTTCCTGTGCGAGTTCCACTTCCCCGCCACCTGTCGGCCCCTGGCCGTG
K A D G F L C E F H F P A T C R P L A V
LysAlaAspGlyPheLeuCysGluPheHisPheProAlaThrCysArgProLeuAlaVal
HisHisHisHisHisHis

1. A diagnostic reader system, comprising a portable reader unit configured to be carried by hand powered by a microprocessor and coupled to a portable computer configured to allow data transfer between said diagnostic reader and said portable computer, wherein said diagnostic reader contains a stylet or other unit to obtain a blood sample by finger stick or other bodily fluid sample and is operably connected to a disposable diagnostic device which has been contacted by the blood or other fluid sample taken from a person who may have suffered a blow or trauma involving the person's head, and wherein said disposable diagnostic device has:
a. at least one first reactive surface which has a first type of biomolecular reagent bonded to it by which to measure a blood-borne concentration of at least one first mitochondrial releasate;
b. at least one second reactive surface which has a second type of biomolecular reagent bonded to it by which to measure a blood-borne concentration of at least one second mitochondrial releasate; and,
c. at least one third reactive surface which has a third type of biomolecular reagent bonded to it by which to measure a blood-borne concentration of at least one third mitochondrial releasate; and wherein said diagnostic reader system has data handling means selected from the group consisting of:
i. displaying, in a manner visible to a user, the blood-borne concentrations of said first, second and third mitochondrial releasates; and,
ii. transferring, to a portable computer, such as a smartphone or tablet, which has a display monitor, the blood-borne concentrations of said first, second and third mitochondrial releasates.

2. The diagnostic reader system of claim 1 wherein said first, second and third mitochondrial releasates are selected from the group consisting of:
a. DNA segments (“native”) which are unaltered and specific to mitochondrial genes (mitochondrial DNA) and which do not normally occur in human nuclear DNA;
b. fragments of mitochondrial DNA that have been degraded by oxidative radicals as a result of the brain injury in ways that normally are found, in humans, only in DNA fragments that have been released by mitochondria;
c. proteins that are encoded by mitochondrial or nuclear DNA and concentrated in mitochondria prior to their rupture by the brain injury, including but not limited to;
i. “high mobility group” ( HMG) proteins;
(a) High mobility group box 1 protein ( HMGB1);
(b) Transcription Factor for Mitochondria A (TFAM);
ii. Cytochrome C oxidase;
iii. Cyclophilin D;
iv. Subunit 6 of ATP synthase;
v. N-formyl peptides (N-FPs) and formyl peptide receptors (FPRs)

3. The diagnostic reader system of claim 1 wherein both of said first and second reactive surfaces are positioned in different locations on the single disposable diagnostic device, separated from the third reactive surface.

4. The diagnostic reader system of claim 1 wherein said diagnostic reader system is also capable of reading data from different disposable diagnostic devices which have distinct areas that have been coated with biomolecular reagents that will indicate concentrations of one or more blood-borne human proteins selected from the group consisting of:
a. apolipoprotein E;
b. apolipoprotein A-1;
c. one or more selected TAR DNA binding proteins;
d. one or more cellular damage-associated molecular patterns (DAMPs) and damage-related proteins selected from the group consisting of;
i. HMGB1 and TFAM (above as mitochondrial DAMPs) and;
ii. angiotensin-converting enzyme serpin proteins, and plasminogen activator inhibitors;
e. cytokines and/or other proinflammatory mediators;
f. mRNA from genes which encode subunits of receptors that interact with cytokines or proinflammatory mediators and that include;
i. thrombomodulin (THBD);
ii. endothelial cell protein C receptor (ECPCR);
iii. 5-hydroxytryptamine receptor 2A;
iv. the serotonin transporter (SERT) or solute carrier family 6 (neurotransmitter transporter, 5-HTT);
v. the human protein designated as SLC6A4;
g. protein fragments normally found in receptors for thrombin and HMGB1 such as thrombomodulin (THBD);
h. protein fragments normally found in receptors for advanced glycation endproducts (AGEs) such the receptor for AGEs (RAGE); and
i. protein fragments normally found in pattern recognition receptors (PRRs) such as soluble Toll-like receptor (sTLR2 and sTLR4).

5. A genetic vector containing an engineered gene sequence which encodes an initial polypeptide which contains:
(a) at least one soluble domain derived from human thrombomodulin;
(b) at least one domain derived from the “Box A” domain of the “High-Mobility Protein Group, Box 1” ( HMGB1) human protein; and,
(c) a secretion sequence which will cause said at least a portion of said initial polypeptide, containing said thrombomodulin and HMGB1 domains, to be secreted by host cells which have synthesized said initial polypeptide; and
(d) wherein said engineered gene sequence contains a plurality of substituted codons which have been selected and inserted into said engineered gene sequence to maximize expression of said initial polypeptide by a selected type of host cell, wherein said substituted codons do not occur in natural human genes which encode said thrombomodulin or HMGB1 domains, but wherein said substituted codons encode the same amino acids as the codons in said natural human genes.

6. The genetic vector of claim 5, wherein said vector can transfect and replicate in human cells and will express said initial polypeptide in a manner which causes said initial polypeptide to be processed and secreted by said human cells in a smaller form which does not contain the secretion sequence.

7. The genetic vector of claim 5, wherein said vector encodes an engineered protein from which one or more cysteine residues, which are not involved in disulfide bond formation, have been replaced by other amino acid residues.

8. A liquid preparation suited for intravenous injection into animals or patients, containing a soluble chimeric protein which contains:
(a) at least one soluble domain derived from human thrombomodulin; and,
(b) at least one domain derived from the “Box A” domain of the “High-Mobility Group Box 1” Protein ( HMGB1) human protein,
wherein said soluble chimeric protein is therapeutically useful for modulating neuro-inflammation.

9. The liquid preparation of claim 8, wherein one or more cysteine residues which are not involved in disulfide bond formation, in native versions of said thrombomodulin and HMGB1 domains, have been replaced by other amino acid residues.


SEQUENCE LISTING (2 sequences)

SEQ ID NO: 1 - DNA, 813 bp, ARTIFICIAL SEQUENCE
  HYBRID
ccgcgctggttacttaagcttggtaccgccaccatggagaccgacaccctgctgctgtgggtgctgctgctgtgggtgcccggcagcaccggcggcaagggcgaccccaagaaacccagaggcaagatgagcagctacgccttctttgtgcagacctgcagagaggaacacaagaagaagcaccccgacgccagcgtgaacttcagcgagttcagcaagaaatgcagcgagcggtggaaaaccatgagcgccaaagagaagggcaagttcgaggacatggccaaggccgacaaggccagatacgagagagagatgaagacctatatcgcccctgccgagcctcagcctggcggctctcagtgcgtggaacacgactgcttcgccctgtaccccggacccgccaccttcctgaacgccagccagatctgcgacggcctgcggggccatctgatgaccgtgcggtctagcgtggccgccgacgtgatcagcctgctgctgaatggcgacggcggcgtgggacggcggagactgtggattggactgcagctgccccctggctgcggcgaccctaagagactgggccccctgagaggcttccagtgggtgacaggcgacaacaacaccagctacagcagatgggccagactggacctgaatggcgcccctctgtgcggccctctgtgtgtggctgtgtctgccgccgaggccaccgtgcctagcgagcccatttgggaggaacagcagtgcgaagtgaaggccgacggcttcctgtgcgagttccacttccccgccacctgtcggcccctggccgtg

SEQ ID NO: 2 - protein, 266 aa, ARTIFICIAL SEQUENCE
  HYBRID
METDTLLLWVLLLWVPGSTGGKGDPKKPRGKMSSYAFFVQTCREEHKKKHPDASVNFSEFSKKCSERWKTMSAKEKGKFEDMAKADKARYEREMKTYIAPAEPQPGGSQCVEHDCFALYPGPATFLNASQICDGLRGHLMTVRSSVAADVISLLLNGDGGVGRRRLWIGLQLPPGCGDPKVGRRRLWIGLQLPPGCGDPKRWARLDLNGAPLCGPLCVAVSAAEATVPSEPIWEEQQCEVKADGFLCEFHFPATCRPLAVHHHHHH